(19) United States
(12) Plant Patent Application Publication
(10) Pub. No.: US 2006/0026724 P1
(43) Pub. Date: Feb. 2, 2006

(54) Pear tree named 'Maxie'

(50) PYRUS COMMUNIS X PYRUS PYRIFOLIA; PEAR

(76) Inventor: Roy Hart, Nelson (NZ)

Correspondence Address: GREENLEE WINNER AND SULLIVAN P C, 4875 PEARL EAST CIRCLE, SUITE 200, BOULDER, CO, 80301, US

(21) Appl. No.: 10/900,691

(22) Filed: Jul. 27, 2004

(51) Int. Cl. A01H 5/00

(52) U.S. Cl. PLT/173

(57) ABSTRACT

A new distinct pear variety is described. This interspecific variety results from crossing varieties Nijisseiki (Pyrus pyrifolia Nakai) (not patented) and Max Red Bartlett (Pyrus communis) (U.S. Plant Pat. No. 741). The new variety has been named ‘Maxie’ and was selected because of its crisp texture and strong ‘Bartlett’ type flavour. The fruit of this new variety is characterised by its round to high round fruit shape which has 50-70% red blush coverage.

### GENUS AND SPECIES OF PLANT CLAIMED

Pyrus communis×Pyrus pyrifolia

### BACKGROUND OF THE INVENTION

A new distinct pear variety is described. This interspecific variety results from crossing varieties Nijisseiki (Pyrus pyrifolia Nakai) (not patented) and Max Red Bartlett (Pyrus communis) (U.S. Plant Pat. No. 741). The new variety has been named ‘Maxie’ and was selected because of its crisp texture and strong ‘Bartlett’ type flavour. The fruit of this new variety is characterised by its round to high round fruit shape which has 50-70% red blush coverage.

### SUMMARY OF THE INVENTION

This new variety was selected from a population of seedlings derived from crossing the pear varieties ‘Nijisseiki’ and ‘Max Red Bartlett’. The cross was made in 1987 at Motueka, Nelson, New Zealand. The variety was determined to be distinct from the parent varieties by the following characteristics:
Nijisseiki: ‘Maxie’ fruit has 50-70 percent red colour while ‘Nijisseiki’ has none; strong ‘Bartlett’ like flavour when fully ripe compared with the delicate flavour of ‘Nijisseiki’; a layer of stone cells around the core while ‘Nijisseiki’ has none. ‘Max Red Bartlett’: ‘Maxie’ has a round to high round shape compared to the pyriform shape of ‘Max Red Bartlett’; ‘Maxie’ has red skin colour and can have a ‘speckled’ appearance where colour is concentrated around lenticels while the colour on ‘Max Red Bartlett’ has fuller skin coverage.
‘Williams Bon Chretien’: ‘Maxie’ has a round to high round shape compared to the pyriform shape of ‘Williams Bon Chretien’; ‘Maxie's’ red skin colour and can have a ‘speckled’ appearance where colour is concentrated around lenticels while ‘William' Bon Chretien’ has no skin colour.

The new variety has been named ‘Maxie’. True to type budding and grafting onto Quince rootstock ‘BA29 with ‘Buerre Hardy’ pear as an interstock has been used to obtain plants. Asexual propagation by budding and grafting has shown that the unique combination of characteristics of the variety is consistently transferred through successive propagation.

### BRIEF DESCRIPTION OF THE DRAWINGS

The accompanying photographs show typical specimens of the tree, foliage, fruit of the new variety as depicted in colours as nearly true as is reasonably possible to make the same colour illustration of this character.

FIG. 1: side view of typical fruit of the pear variety ‘Maxie’.

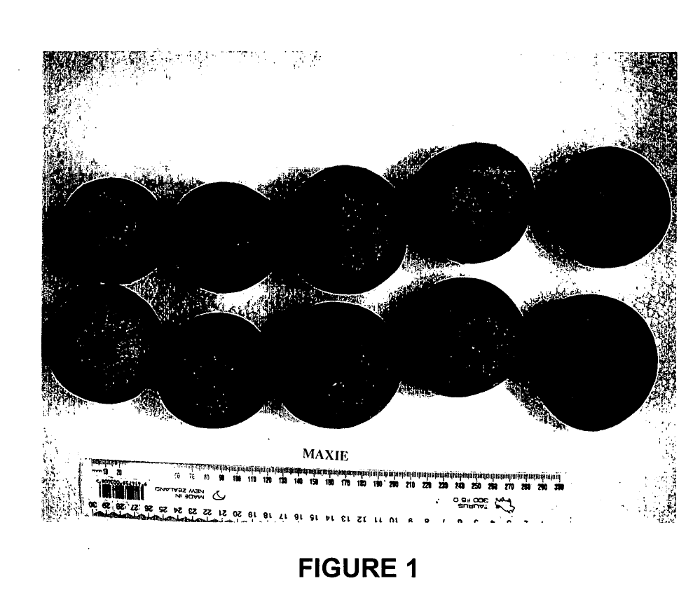

FIG. 2: calyx end view of typical fruit of the pear variety ‘Maxie’.

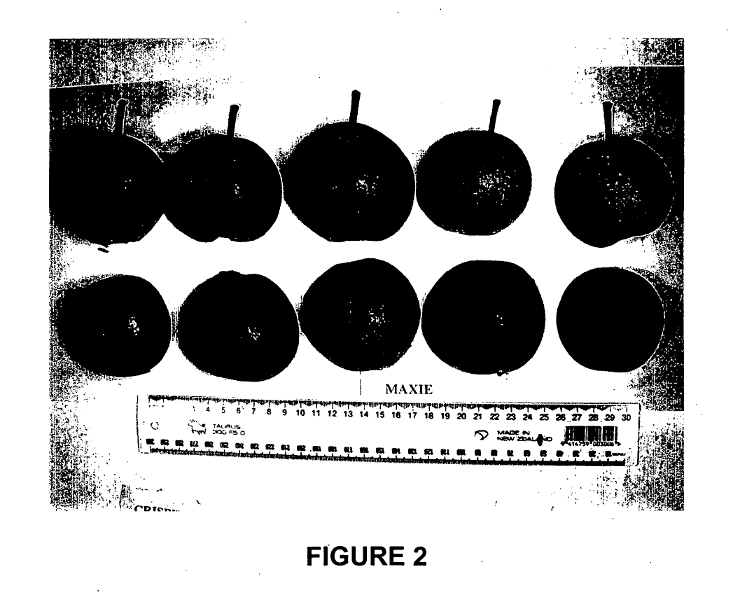

FIG. 3: stem end view of a typical fruit of the pear variety ‘Maxie’.

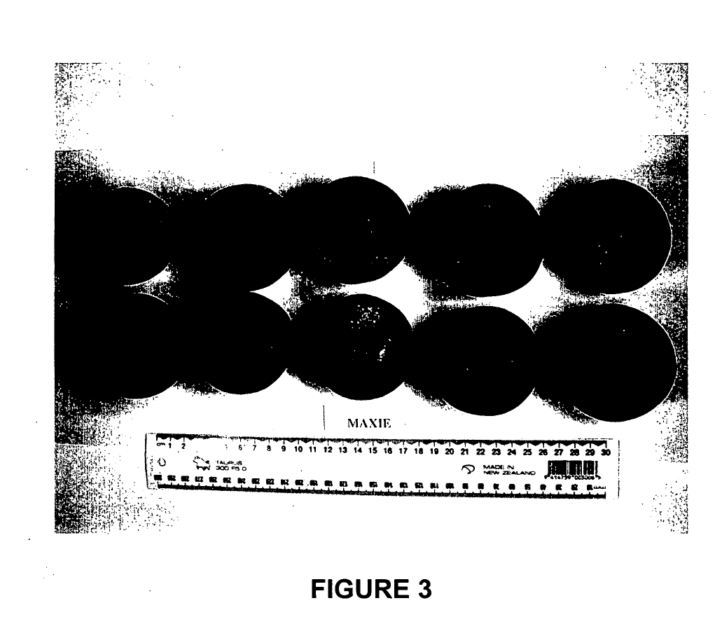

FIG. 4: cross sectional view of a typical fruit of the pear variety ‘Maxie’.

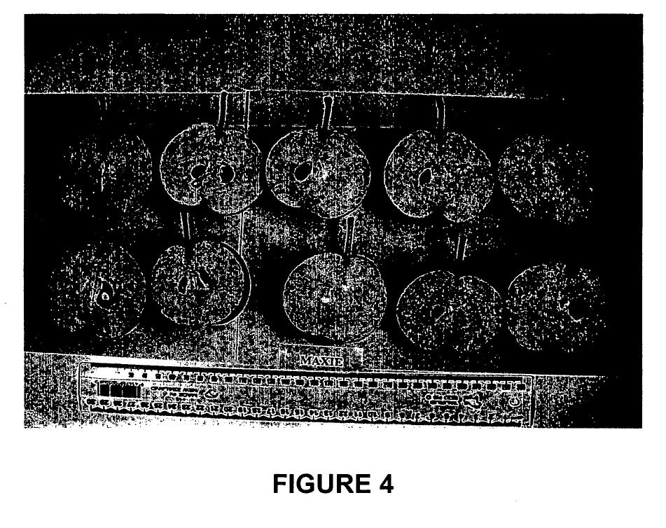

FIG. 5: typical mature leaves of ‘Maxie’ showing the upper and lower surfaces.

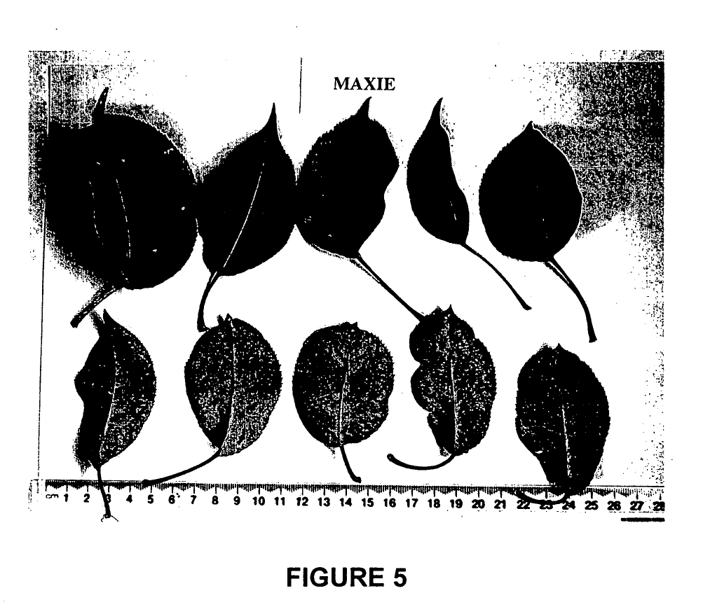

FIG. 6: cross sectional fruit view of ‘Nijisseiki’, (far left), ‘Maxie’ (middle), ‘Williams' Bon Chretien’ (right).

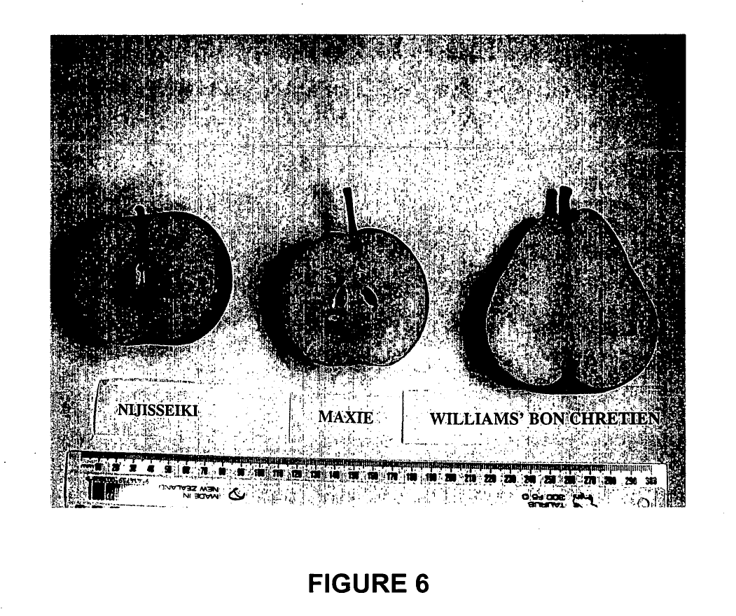

FIG. 7: fruiting spur ‘Maxie’.

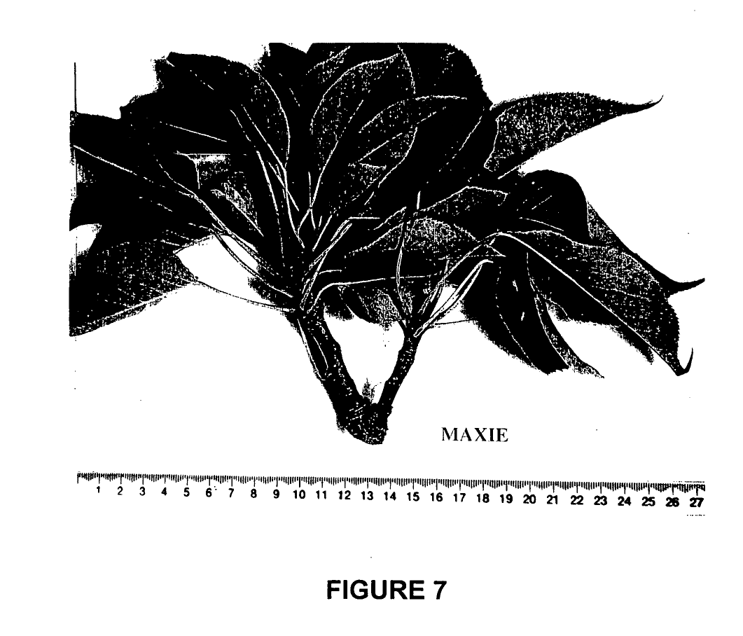

### MORPHOLOGICAL DESCRIPTION OF THE VARIETY

The following is a detailed description of the new variety with colour terminology in accordance with The Royal Horticultural Society Colour charts (R.H.S.C.C.) 2001 edition.

The observations were made in the 2003-2004 season on trees grafted onto Quince ‘BA29’ rootstocks with a ‘Buerre Hardy’ pear interstock and planted in the orchard in 1999.
Tree: Vigour when grafted onto Quince ‘BA29’ rootsocks with a ‘Buerre Hardy’ interstock weak; size medium small; semi upright; branching weak; bearing on spurs which can become multi spurred.
Trunk: Bark is grey R.H.S.C. C. N200D when mature; trunk circumference of 162 mm at a height of 200 mm.
Branches: Thin; weak, little branching, approximately 4 spurs/100 mm of 2 year old wood; low annual growth numbers with a length of 400-500 mm; annual growth wood brown purple R.H.S.C.C. N77A.
Lenticels: Medium numbers of large sized lenticels on current years growth.
Leaves: Average length 60 mm; width 48 mm; length/width ratio 1.24; downward pose, upfolded shape in cross section; indentations of the margin bluntly serrate, medium depth; shape of leaf blade apex (excluding pointed tip) obtuse; petiole length 36-51 mm; stipules absent; colour of leaf blade green R.H.S.C.C. 146A
Flower buds: Flower bud length medium short; stigma can protrude above flower bud petals.
Flowers: Bloom period medium—late, starting 25 September until 15 October in Nelson New Zealand, full bloom date 4 October; sepal length medium; sepal attitude in relation to the corolla spreading; margins of petals overlapping; position of stigma in relation to stamens above; size of petal medium; shape of petal (excluding claw) circular; shape of petal base (excluding claw) cordate; length of claw petal short.
Fruit: Fruit is mature for consumption in early March; harvest period is from 20 February to 5 March in Nelson, New Zealand.
Fruit size: Medium small; average fruit weight 160 g; average width 72 mm; average height 66 mm; fruit length/diameter ratio small; maximum diameter in middle of fruit.
Stalk cavity: Shallow, average 4 mm.
Shape: Round—high round; slightly asymmetric in longitudinal section; profile of the sides convex.
Eye basin: Average width 26 mm; average depth 7 mm, relative area of russet around eye basin absent or very small.
Stalk: Thick, length medium, average 26 mm; curvature of stalk absent or very weak, attitude of the stalk is straight in relation to the axis of the fruit; depth of stalk cavity average 3.9 mm.
Sepal: Calyx or sepals are partially persistent, approximately 40% of fruit at harvest have a deciduous calyx and sepals, remaining 60% have erect to spreading sepals.
Skin: Smooth; greasiness absent, cracking tendency of skin absent; thick; background colour, yellow R.H.S.C.C. 7D; lenticels medium.
Overcolour: 50-70% of skin surface red R.H.S.C.C. 46A, amount of russet around the stem cavity absent to low.
Texture: Crisp; flesh medium; firmness medium; flesh juicy; medium numbers of large stone cells around the core cavity.
Flavour: Sweetness medium; acidity low; strong ‘Bartlett’ like aroma when ripe.
Quality: Very good
Seeds: Locules 5, seeds per locule 1-2; medium seed size 11 mm long, 5 mm wide; colour, black R.H.S.C.C. 202A.
Use: Dessert.
Production: Mid season, regular cropping when thinned.
Management: Thinning of crop load in early summer is recommended. Minimal pruning is required because spur bearing habit and the limited amount of one year wood produced.
Pest and disease: Susceptible to pear slug (Caliroa cerasi). Highly leaf and fruit resistant to pear scab (Venturia pirina Adherh).

I claim:

1. A new and distinct variety of pear tree named ‘Maxie’ substantially as herein illustrated and described.
